(19) United States
(12) Patent Application Publication
(10) Pub. No.: US 2017/0202692 A1
(43) Pub. Date: Jul. 20, 2017

(54) A DEVICE TO ASSIST IN SELF INSERTION OF A CATHETER TUBE INTO THE URETHRAL ORIFICE OF WOMEN

(71) Applicant: GR Dome Medical Ltd., Tirat Carmel (IL)

(72) Inventor: Amir LANIADO, Haifa (IL)

(21) Appl. No.: 15/512,557

(22) PCT Filed: Oct. 7, 2014
(86) PCT No.: PCT/IL2014/000051

§ 371 Date: Mar. 19, 2017

(51) Int. Cl. A61F 5/00; A61F 5/451; A61F 5/44; A61F 5/455

(52) U.S. Cl. CPC A61F 5/00; A61F 5/4553; A61F 5/455; A61F 5/451; A61F 5/44; A61M 25/0017

(57) ABSTRACT

The present invention is a device to assist in the insertion of a catheter tube into the urethral tract of women The device simplifies the finding of the location of the external urethral orifice and widens the orifice so that women patients may perform self-insertions of a catheter into the external orifice of the urethral tract for medical treatments without the assistance of a physician or a caregiver and with substantialy reduced discomfort. The device is constructed of: a vaginal insertion portion, a catheter tube guiding portion, at least two wing structures configured to widen the urethra orifice and a vaginal insertion element. Both the vaginal insertion portion and the catheter tube guiding portion of the UCAD are constructed as plates having elongated configurations. The vaginal insertion portion and the catheter tube guiding portion connect at one of their edges in a spatial configuration that forms an L shaped structure. The vaginal insertion portion of the UCAD has a configuration designed to be placed in the vagina, adjacent to the pubic symphysis of a treated women patient. The catheter tube guiding portion has a hole at the unconnected edge. Through the hole the catheter tube is inserted into the urethral tract of a treated patient. The catheter tube is removable from the hole in the catheter tube guiding portion without having to remove the catheter tube from the orifice of urethral tract, as explained below. The vaginal insertion element is connected to the vaginal insertion portion. The wing structures connect to the catheter tube guiding portion and protrude from the rim of said hole in the catheter tube guiding portion.

### FIELD OF THE INVENTION

The present invention is a device to assist in the insertion of a catheter tube into the urethral tract of women. More specifically, the device of the present simplifies the finding of the location of the external urethral orifice and widens the orifice so that women patients may perform self-insertions of a catheter into the external orifice of the urethral tract for medical treatments without the assistance of a physician or a caregiver.

### BACKGROUND OF THE INVENTION

The term catheter is defined by the Merriam-Webster dictionary (http://www.merriam-webster.com/dictionary/catheter) as: a thin tube that is put into the body to remove or inject a liquid or to keep a passage open. In the context of the present invention the terms: “tube”, “catheter” and “catheter tube”, refer interchangeably to a thin tube that is inserted through the external orifice of the urethra of women and is utilized for the input of medication into the urethra tract and/or bladder and/or for the removing of urine from the bladder. In the text that follows the device of the present invention to assist in the insertion of a tube into the urethral tract in women is also referred to as: “the urethra catheter applicator”, or, interchanchably, as: “the urethra guide”.

Conditions such as cystitis, bladder pain syndrome, interstitial cystitis, urethritis, urethra pain syndrome are inflammatory conditions of the submucosal and muscular layers of the bladder without infectious pathogens. The cause of lower urinary tract inflammation is often, but not necessarily, triggered by an infection (bacterial cystitis) is unknown in many patients and the condition is regarded as a diagnosis of exclusion. Inflammation of the lower urinary tract inflammatory conditions is associated with urinary urgency, urinary frequency, waking at night to urinate (nocturia), and pain/discomfort. Symptoms may overlap with other urinary bladder disorders such as: urinary tract infection (UTI), overactive bladder and prostatitis.

Bladder instillation of pharmaceutical agents is one of the main forms of treatment of lower urinary tract inflammation. Advantages of this treatment approach include direct contact of the medication with the bladder wall and decreased risk of systemic side effects. Bladder instillations use a transurethral approach where a drug is instilled directly into the bladder using a catheter tube on several occasions. Because of the unaccessible position of the urethra in women it is difficult for most women patients to self-administer the bladder instillation.

Patients that suffer from urinary retention problems or from inability to completely empty their bladder during urinating are often treated intermittently with a catheter. The ability to self-insert a catheter tube through the urethra orifice frees a treated patient of relaying on assistance from a medically trained person which is not always convenient or available. Self-insertion of a catheter tube requires fine motor skills and a good vision to locate and penetrate the urethra orifice.

In women, the urethra tract is short compared to males and located at the bottom of the pelvis. The urethra tract is about 3.5-5 cm long and exits the body between the clitoris and the vagina. The women external urethral orifice is located 1-2 cm below the clitoris behind the symphysis pubis. The entire length of the urethra tract is embedded in the anterior vaginal wall and it is slightly curved with the concavity directed downwards. Because of the anatomic boundaries many patients requiring bladder instillations find it difficult to pass the catheter through the urethra tract into the bladder and most local treatment regimes require the patient to return to the physician or trained caregiver repeatedly or undergo training to manage it by themselves.

Prior art devices for self-insertion of catheter-tubes are commonly constructed of two connected portions: a vaginal insertion portion and a catheter tube guide portion. The vagina inserted portion stabilizes and maintains the catheter tube guiding portion in a placement which enables the insertion of the catheter tube through the hole into the urethra orifice. Examples of self-insertion devices of a catheter tube for women that have two portion units are given in U.S. Pat. No. 5,045,078 by Asta and U.S. Pat. No. 5,084,036 by Rosenbaum:

Asta discloses a device that has a vaginal insertion portion and a catheter tube guide portion that has a handle protruding from it. The guiding portion has at least one hole running through it that is alienable with urethra orifice when the inset portion is in the vagina. The device does not include means of widening the orifice of the urethra nor does it allow for the removing the device from the body of the patient while maintaining the catheter in the urethra tract.

Rosenbaum discloses a device that is constructed of a vagina insertion portion and a catheter tube guide portion (referred to as “locating member”) which has a hole running through it (referred to as “passageway) and has a handle protruding from it. A special measuring device is required for constructing the self-insertion catheter tube device. As with Asta's device, Rosenbaum does not include means of widening the urethra orifice nor does it facilitates the removing the device while maintaining the catheter in the urethra tract.

Another approach of self-insertion devices of a catheter tube for women is disclosed by U.S. Pat. No. 7,104,980 by Laherty et al. in which the device includes a main body portion configured to be placed adjacent to the pubic bone of the woman. A pair legs extend downwards from the main body portion with a slot between the legs and a flange extending longitudinally along each leg. In deployment of the device, the location of the connection place of the two legs is positioned over the urethra orifice of the patient and the catheter tube is inserted between the two legs into the orifice with the labia lips spread by the flanges (not aimed at widening the orifice of the urethra) . The device is held from the direction of the belly, thus the device does not enable stability and precision in inserting the catheter tube.

The disclosed devices for assisting women in self-insertion of a catheter tube are designed to insert a catheter into the urinary tract while maintaining the device connected to the body of the treated patient. The continued presence of a device for assisting in self-insertion of a catheter connected to the treated patient after the insertion is complete may cause irritation and/or a discomfort able feelings.

The device of the present invention for assisting in the insertion of a catheter tube into the external orifice of the urethral tract in women, also referred to as: an urethra catheter-applicator device for women and, interchangeably abbreviated to: UCAD, facilitates easy finding of the location of the external urethral orifice and assists in the procedure of the insertion of a catheter into the urethra orifice while widening the orifice. In addition, the UCAD of the present invention enables the user to disconnect the device from her body while maintaining the catheter tube in the urethra tract after it has been inserted. In addition, the UCAD of the present invention is designed as a compact, user friendly, device that is easy to carry and simple to deploy by using a reversibly connected vaginal insertion element.

The UCAD of the present invention enables women patients to perform themselves the instillation of medicinal substances for the treatment of inflammation along the uretheral tract bladder and/or removal of urine from the bladder without the assistance of a physician or a trained caregiver while substantialy reducing the discomfort caused to the patient in the course of performing the medical procedure.

### SUMMARY OF THE INVENTION

In the context of the present invention the terms “tube”, “catheter” and “catheter tube” are used interchangeably and refer to a thin tube that is inserted through the external orifice of the urethra of women and is utilized for the input of medication into the urethra tract and/or bladder and/or for the removing of urine from the bladder. The present invention is a device for assisting women to self-insert into the urethra orifice a catheter tube required for medical treatment, also referred to in the text that follows as UCDA. The device eases/simplifies the procedure of locating the orifice of the urethra and widens the orifice, thus, making the insertion of a catheter tube easier and substantially less discomforting. In addition, the device of the present invention provides women with the option of removing from their body the self-inserting device after the catheter tube has been inserted, leaving the catheter tube inserted into the urethra tract, thus, providing treated women with the ability to choose if to continue to have a device connected to her body when and if the device causes discomfort and/or irritation.

The UCDA of the present is constructed of : a vaginal insertion portion, a catheter tube guiding portion, at least two wing structures configured to widen the urethra orifice and a vaginal insertion element. Both the vaginal insertion portion and the catheter tube guiding portion of the UCAD are constructed as plates having elongated configurations. The vaginal insertion portion and the catheter tube guiding portion connect at one of their edges in a spatial configuration that forms an L shaped structure. The vaginal insertion portion of the UCAD has a configuration designed to be placed in the vagina, adjacent to the pubic symphysis (also referred to as: pubic bone) of a treated women patient. The catheter tube guiding portion has a hole at the unconnected edge. Through the hole the catheter tube is inserted into the urethral tract of a treated patient. The catheter tube is removable from the hole in the catheter tube guiding portion without having to remove the catheter tube from the orifice of urethral tract, as explained below. The vaginal insertion element is connected to the vaginal insertion portion. The wing structures connect to the catheter tube guiding portion and protrude from the rim of said hole in the catheter tube guiding portion.

Furthermore, the hole in the catheter tube guiding portion has a gap in the circumference of the hole that leads to the external surrounding of the catheter tube guiding portion. Through the gap, by squeezing and compressing the catheter tube in the near of the gap, the tube is slipped through the gap and removed from the hole.

Furthermore, the UCAD alternatively includes a hole closing bar element that reversibly connects to the edge of the catheter tube guiding portion not connected to the vaginal insertion portion. The reversible connecting of the closing bar element defines (forms) a completely encircled hole at edge of the catheter tube guiding portion. By disconnecting the closing bar element, the defined encircled hole is no longer intact and the catheter tube is removable from the hole of the catheter tube guiding portion.

Furthermore, the vaginal insertion portion and the catheter tube guiding portion of the UCAD are produced in a connected configuration.

Furthermore, vaginal insertion portion and the catheter tube guiding portion of UCAD are alternatively produces with each portion having a separate entity and are connected together to conform a single entity.

Furthermore, the vaginal insertion portion and the catheter tube guiding portion of UCAD connect in a fixed angle of approximately ninety degrees. Alternatively the vaginal insertion portion and the catheter tube guiding portion connect in an adjustable fixated angle.

Furthermore, the wing structures wing structures of UCAD that are configured to widen the urethra orifice, are produced as an integral part of the catheter tube guiding portion. Alternatively, the wing structures are connected to catheter tube guiding portion by hinges.

Furthermore, the vaginal insertion element of UCAD reversibly connect to the vaginal insertion portion. The vaginal insertion element can be of any configuration that enables the simple and comfortable insertion of the vaginal insertion portion of UCAD into the vagina of the treated patient, such as an insertion thimble and an insertion thimble and an insertion rod-handle.

In the process of deployment, the vaginal insertion portion of the UCAD is inserted by the user into the vagina between the lips of the labia minora that are parted by one hand by the patient. The insertion is done by pressing the vaginal insertion element towards the vagina with the other hand. The inserted vaginal insertion element portion is pressed towards the pubis symphysis, thus stabilizing the device in place. In the insertion of the vaginal insertion element into the vagina, the wing-constructions of the catheter tube guiding portion are guided to connect to the skin surrounding orifice of the urethra and stretch-widen the orifice. The widened orifice is aligned with the hole in the guiding portion of the catheter tube guiding portion. The patient inserts a catheter tube through the hole and through the widened orifice of the urethra. With the catheter tube inserted into the body of the patient, the patient is at liberty to disconnect UCAD from her body while maintaining the catheter tube inserted in her urethra tract.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals. The urethra catheter-applicator device for women for self-insertion of a catheter tube of the present invention of the present invention is also referred interchangeably as UCAD.

FIG. 1 illustrates an isometric from-above-and-side view of a UCAD shown in a disassembled configuration. Also shown: a disconnected closing-bar element and two alternative vaginal insertion elements, an insertion-thimble and an insertion rod-handle.

FIG. 2 illustrates from-above-and-side isometric view of the UCAD shown in FIG. 1 in an assembled, closed by the closing-bar element, configuration and connected to the insertion thimble.

FIG. 3 illustrates from-above-and-side isometric view of the UCAD shown in FIG. 1 in a an assembled, closed by the closing-bar element, configuration and connected to the insertion rod-handle.

FIG. 4A illustrates from-above-and-side isometric view of the UCAD shown in FIG. 1, with wings with hinges for widening the external urethra orifice of a treated patient, in a disassembled configuration, and without an insertion rod-handle or an insertion thimble.

FIG. 4B illustrates from-above-and-side isometric view of the UCAD shown in FIG. 4A, with wings with hinges in an assembled configuration.

FIG. 5 to and including FIG. 9, illustrate stages of deploying the UCAD, shown in FIG. 1 and FIG. 2, in a treated patient.

FIG. 5 illustrates the connecting procedure by a treated patient of the UCAD shown in FIG. 1 to an insertion thimble. The UCAD is shown in a configuration closed by a closing-bar element.

FIG. 6 illustrates the inserting into the vagina of the UCAD, shown in FIG. 5.

FIG. 7 illustrates the inserting of a tube of a catheter trough the UCAD shown in FIG. 5, with the device inserted in the vagina of the treated patient.

FIG. 8 illustrates the removing of the UCAD shown in FIG. 5 from the body of the treated patient.

FIG. 9 illustrates a catheter tube remaining inserted into the body, with the UCAD removed from the body of the treated patient.

FIG. 10 illustrates a UCAD with an inserting rod handle and without a closing bar element, inserted into the vagina of a treated patient.

FIG. 11 illustrates the removing of the UCAD shown in FIG. 10 from the body of the treated patient.

FIG. 12 is a cross cut, from the side, illustration of internal organs of a patient with the UCAD shown in FIG. 2 deployed in the body of a treated patient.

FIG. 13A is a cross cut, from the front, illustration of the UCAD shown in FIG. 2 deployed in a treated patient, showing initiation of contact with the skin surrounding the urethra orifice of the patient.

FIG. 13B is a cross cut illustration of the UCAD shown in FIG. 13A showing the contact with the skin surrounding the urethra orifice and widening of the orifice of the urethra of the treated patient

### DETAILED DESCRIPTION OF A PREFERRED EMBODIMENT

To describe an embodiment of the present invention of an urethra catheter-applicator device (abbreviated: UCAD) for women for assisting in self-insertion of a catheter tube into the urethra tract, reference is presently made to the figures.

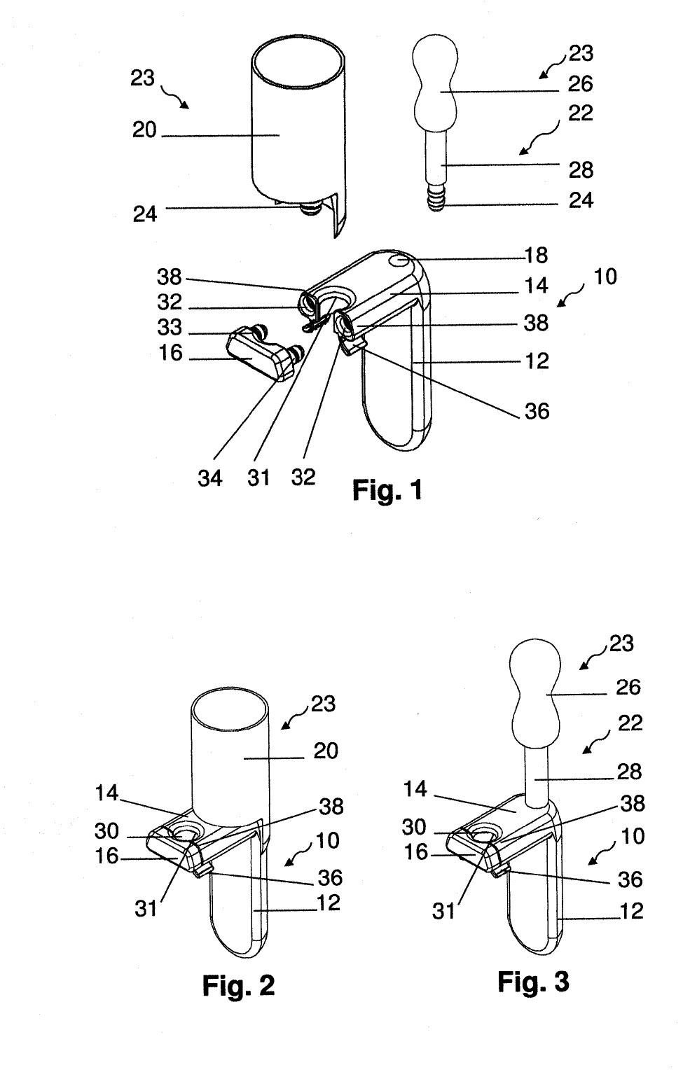

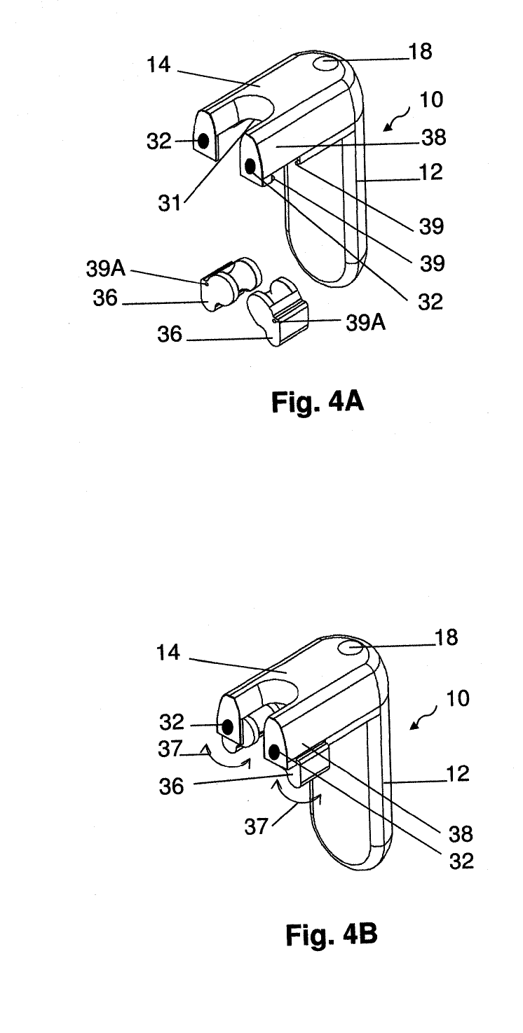

From FIG. 1 up to and including FIG. 4B, reference is made to the construction of the device of the present invention.

FIG. 1 illustrates an isometric from-above-and-side view of a UCAD (10) shown in a disassembled configuration. The UCAD (10) is shown is shown in an opened hole configuration with hole closing-bar element (16) (also referred to as: closing element) adjacent to the UCAD (10). Also shown in the figure are two alternative vaginal insertion elements: an insertion-thimble (20) and an insertion rod-handle (22).

The UCAD (10) and closing element (16) are typically but not necessarily, made of a rigid or semi-rigid material such as, but not limited to silicon, plastic or nylon. The UCAD is typically, but not necessarily, constructed as a single entity that is divided into two elongated typically, but not limited to, rectangle plate-portions: a vaginal insertion portion (12) and a catheter tube guiding portion (14). The term “vaginal insertion portion” (12) is interchangeably used in the context of the present text with the term “insertion portion”. The term “catheter tube guiding portion” (14) is interchangeably used in the context of the present text with the term “guiding portion”. The two portions connect to each other at a fixed angle of, but not limited to, approximately 90 degrees, forming between them an approximate “L” shaped construction, (12) being the long arm and (14) the short arm of the “L”, respectively. Alternatively, the angle between the two portions can be reversibly fixated in accordance with the desire of the treated patient. The terms “inward” and “outward” sides of the plates of the “L” configuration are used in the text for describing the sides that are confined by. the L (the sides forming a semi rectangle frame configuration) or not confined by the “L”, respectively.

The configuration description of the rectangle plate-portions of the UCAD that follows provide a description of typical spatial configuration structures and in no way limit the spatial configuration structures of UCAD plate-portions and the hole closing-bar element of the present invention in having other spatial two-plates, “L” configuration, structures.

The vaginal insertion portion (12) connects at one end to the catheter tube guiding portion (14) and has a smooth and curved edge at its other end. The inward surface of the plate of insertion portion (12) is flat and smooth. The outward surface of the insertion portion (12) has a smooth, conveyed, half-tube configuration that runs along the entire length of the plate.

The catheter tube guiding portion (14) is typically constructed of a rectangle plate having in its inward surface a concaved canal that occupies approximately half the surface area of the plate and runs along the entire length of the plate. Flat margin surfaces run along the entire length of the margins of the canal. On the outward surface of device (10), in the vicinity of the connection plane between the plates of insertion portion (12) and catheter tube guiding portion (14), is a hole (18) used for the reversible connection to either insertion-thimble (20) or, alternatively, insertion rod-handle (22) to the UCAD.

The edge of the plate of catheter tube guiding portion (14) that is not connected to the plate of vaginal insertion portion (12), has an open-ring structure (31) that has on each of its sides in the plane of the plate, a hole (32). Holes (32) are constructed in the margin components (38). A UCAD closing- element (16) typically made of, but not limited to, the same material the UCAD (10), is constructed of bar that one of its edges has an open-ring structure (33) that complements the open-ring structure (31) at the edge of catheter tube guiding portion (14). The bar of UCAD closing element (16) has on each of the sides of the open-ring formation, in the plane of the open-ring formation, a protruding bar (34) that is designed to reversibly penetrate and connect to hole (32) in catheter tube guiding portion (14). The hole (30) defined (formed) when the UCAD closing element (16) is connected to the plate of catheter tube guiding portion (14), is designed to enable the guided insertion of a catheter tube through catheter tube guiding portion (14) and into the orifice of the urethra of the treated patient. By disconnecting UCAD closing element (16) from the plate of catheter tube guiding portion (14), by removing at least one of bars (34) from at least one of holes (32), a catheter tube that is inserted into the urethra orifice and encircled by hole (30) in tube guiding portion (14) can be removed from the UCAD (10) while still remaining inserted through the urethra orifice of the treated patient.

Alternatively to forming hole (30) at the end of catheter tube guiding portion (14) by reversibly connecting guiding portion (14) to open-ring structure hole closing element (16) by bars (34), closing element (16) can be reversibly connected to guiding portion (14) by various connecting mechanisms such as, but not limited to, sliding bars or springs that originated from margin components (38) and connect to closing element (16).

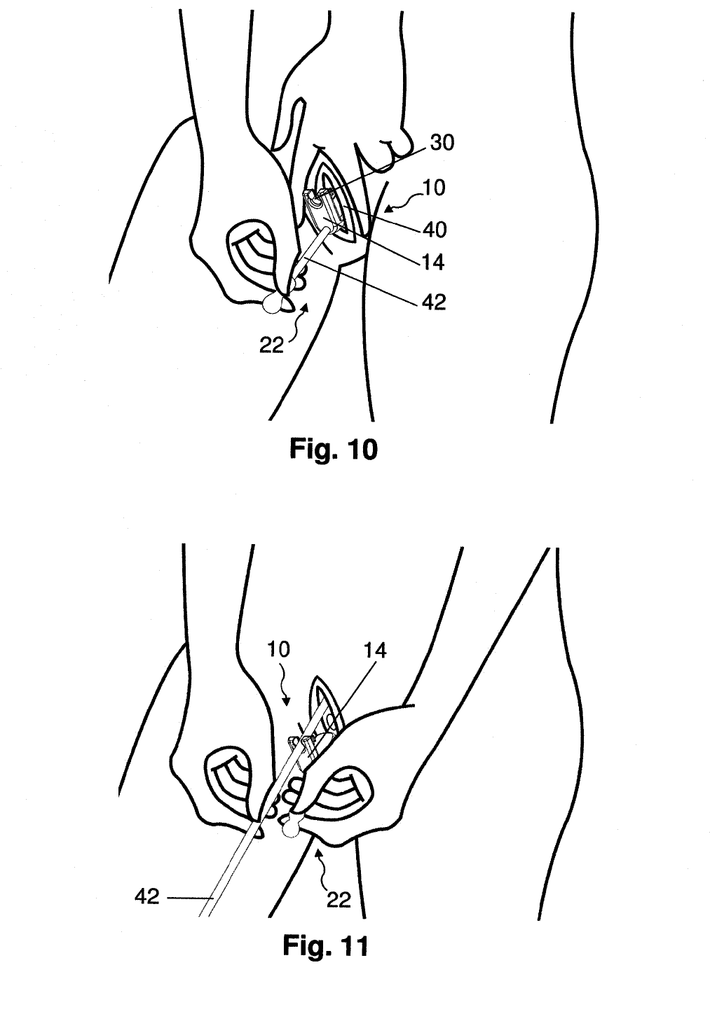

Alternatively to forming hole (30) at the end of catheter tube guiding portion (14) by reversibly connecting guiding portion (14) to open-ring structure hole closing element (16) in the UCAD (10) shown in FIG. 10 and FIG. 11, at the vicinity of the edge of catheter tube guiding portion (14) is a hole with a gap in its circumference. The hole is engulfed by the plate of guiding portion (14) and no open-ring structure hole closing element (16) is utilized. The gap forms a free passage from inside the hole to the external edge, outside of the hole, of plate (14). The width of the gap is construct so as enable the withdrawing of a catheter tube through the gap from the hole, by squeezing and compressing the width of a catheter tube in the near vicinity of the gap.

In the inward side of the edge of catheter tube guiding portion (14), on the margin components that define the open-ring structure (38), are at least two protruding rigid or semi rigid wing structures, one on every margin component. The wing structures (36) are either an integral part of the construction of the UCAD (10) or, alternatively, they are separate entities and are connected to the plate of catheter tube guiding portion (14) by hinges (shown in FIG. 4B). Alternatively the wing structures (36) are connected to the catheter tube guiding portion (14) by, but not limited to, resilient springs that are not an integral part of catheter tube guiding portion (14), The wing structures typically (36) have, but not limited to, a curved plate configuration designed to widen the opening of the external orifice of the urethra when the UCAD (10) is deployed. The wing structures are constructed of the same material that the UCAD (10) is constructed of, or, alternatively, they are produced from any rigid and/or semi-rigid material or a combination of materials that facilitates the widening the opening of the orifice of the urethra and is in accepted for use in coming in contact with human sensitive skin.

Reference is presently made to the two alternative vaginal insertion elements (23) shown in FIG. 1: insertion-thimble (20) and insertion rod-handle (22). The configuration of the vaginal insertion element (23) of the present device is not limited to the two described elements and may be of any configuration that facilitates the insertion and removal of the vaginal insertion portion (12) into the vagina of the treated patient.

Insertion-thimble (20) is typically, but not necessarily, made as single entity component, and is constructed of a rigid or semi-rigid material such as, but not limited, to silicon plastic or metal. The insertion-thimble (20) is constructed of a thin-walled tube opened at one end and sealed at its other end by a sealing-plate surface. The external surface of the sealing-plate has a protruding connecting-rod (24) that is designed to reversibly penetrate and connect to hole (18) in the plate of catheter tube guiding portion (14).

Insertion rod-handle (22) typically, but not necessarily, made as single rod entity component, and is constructed of a rigid material or materials, such as, but not limited, to silicon, plastic or metal. Insertion rod-handle (22) is constructed of a hand-gripping portion (26), an extension-rod (28) and a connecting-rod (24) portion. Connecting-rod (24) is designed to reversibly penetrate and connect to hole (18) in the plate of catheter tube guiding portion (14).

FIG. 2 and FIG. 3 illustrate from-above-and-side isometric views of the UCAD (10) shown in FIG. 1 connected to an insertion-thimble (20) and to an insertion rod handle (22), respectively. In both the figures the UCAD closing element (16) is shown connected to the plate of catheter tube guiding portion (14).

FIG. 4A and FIG. 4B illustrate from-above-and-side isometric views of the UCAD shown in FIG. 1, with wing constructions (36) that are connected to the plate of catheter tube guiding portion (14) of the UCAD (10) by a hinge connection. Hinge-pins (39) are inserted into holes (39A) in wing constrictions (36). The connection of wing constructions (36) to the plate of catheter tube guiding portion (14) is either by a fixed and rigid connection (illustrated in FIG. 1), or alternatively, as illustrated in the present figures, a connection that facilitates the reversible connection and removal of the wing constructions and facilitates side-way motions, as illustrated in FIG. 4B. Wing constructions (36), either in a fixed of or in motion-able connection to catheter tube guiding portion (14), contact the skin surrounding the orifice of the of the urethra and pull the skin sideways, thus, widening the urethra orifice while stabilizing UCAD in its contact with the body of the treated patient. The contact of the skin surrounding the orifice of urethra tract with hinge-connected wing constructions is more graduated and softened in comparison to connection formed by fixe-connection (un-hinged) wing constructions (36).

FIG. 4A illustrates the wing constructions (36) disconnected from the hinge-pins (39) of plate of catheter tube guiding portion (14). FIG. 4B illustrates the wing constructions (36) connected to the plate of catheter tube guiding portion (14). The Double-headed arrows (37) indicate the side-movement of wing constructions (36) facilitated by the hinges.

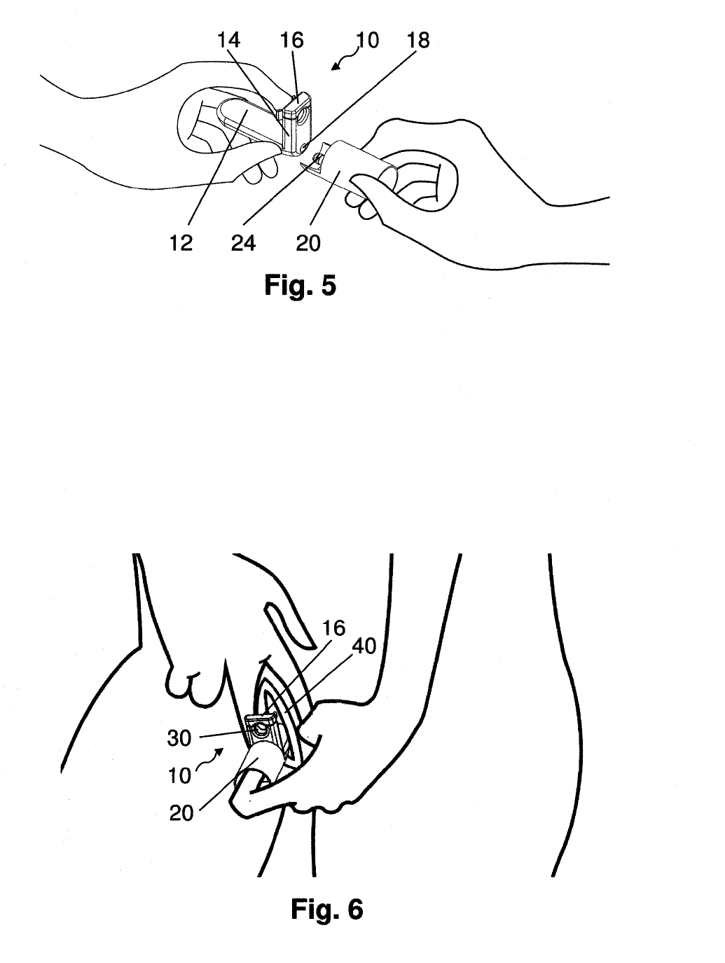

Reference is presently made to the sequence of figures FIG. 5 up to and including FIG. 9, which illustrate the sequence of the activities in deploying a UCAD of the present invention.

FIG. 5 illustrates the connecting procedure of the UCAD (10) shown in FIG. 1 to an insertion thimble (20). The UCAD (10) is shown in a closed configuration, with closing element (16) connected to catheter tube guiding portion (14). Holding the UCAD (10) in one hand and the insertion thimble (20) in the other hand, the patient inserts rod (24) into hole (18), thus forming a reversible yet stable connection between the UCAD (10) and the insertion thimble (20). Alternatively, instead of connecting an insertion thimble (20), an insertion rod-handle (22), shown in FIGS. 3, 10 and 11, is connected to the UCAD (10).

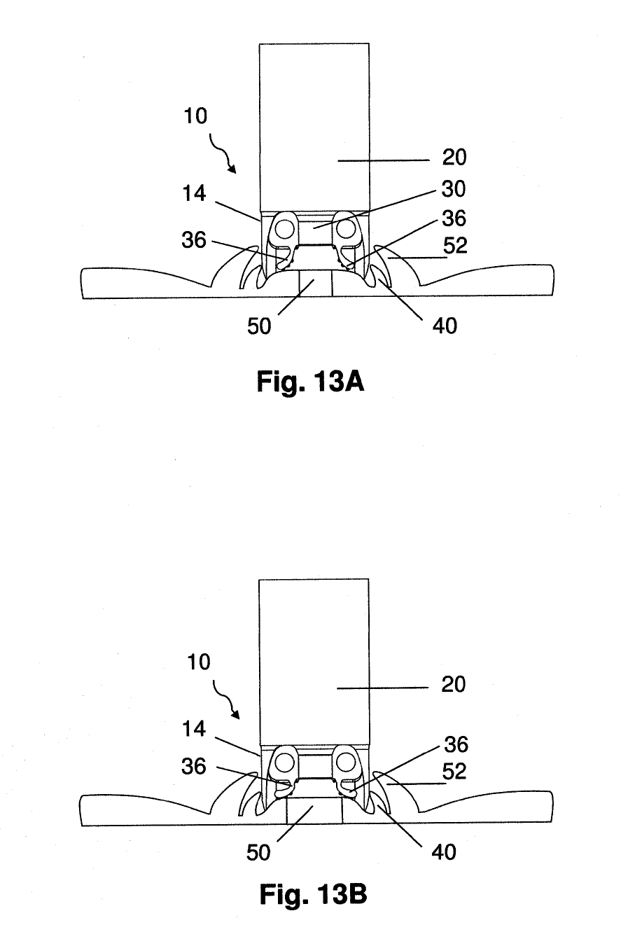

FIG. 6 illustrates the inserting into the vagina of the UCAD (10). The patient spreads her labia minora lips (40) with one hand and inserts a finger of her other hand into the insertion thimble (20). By pushing with her finger that is inserted in the insertion thimble (20), the patient inserts the plate of insertion portion (12) of UCAD (10) into the vagina and presses the plate towards the pubic symphysis, thus, causing the tube guiding portion (14) to be pressed to the upper region of the labia minora lips (40) and having hole (30) adjacent to the urethra orifice of the patient. By pressing the plate of insertion portion (12) of UCAD (10) into the vagina and pressing the plate towards the pubic symphysis, wing structures (36) widen the orifice of the urethra by contacting the skin surrounding the orifice and pushing it side-ways (shown in FIG. 13B).

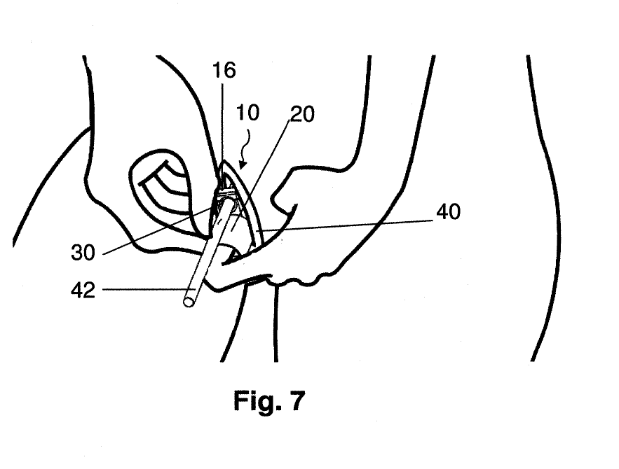

FIG. 7 illustrates the inserting of catheter tube (42) through the hole in the catheter tube guiding portion (14) of UCAD (10). The patient is shown maintaining the plate of insertion portion (12) of UCAD (10) in her vagina with one hand and inserting a tube (42) of a catheter into hole (30) and into the orifice of the urethra with the other hand.

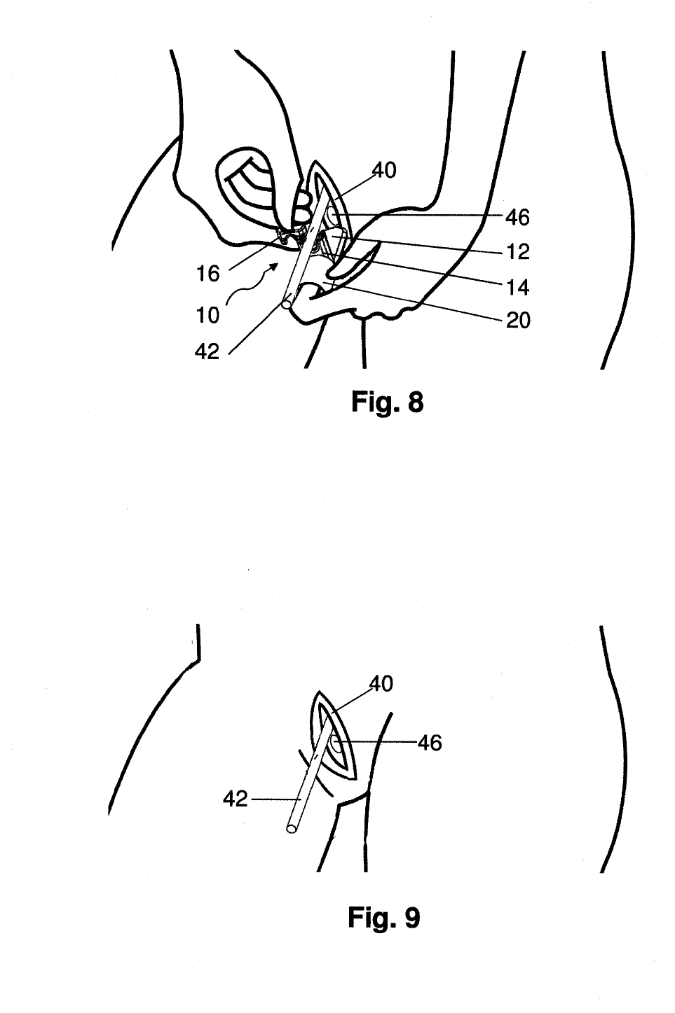

FIG. 8 illustrates the removing of the UCAD (10) from the body of a treated patient. The patient is shown removing the insertion portion (12) of UCAD (10) from her vagina with one hand releasing closing element (16) from the connection with the plate of catheter tube guiding portion (14) of UCAD (10) with the other hand, thus enabling the removal of the insertion portion (12) of UCAD (10) from the vagina, and the UCAD from the body of the patient, while keeping the catheter tube, inserted into the urethra tract of the treated patient, as illustrated in FIG. 9.

Reference is presently made to FIG. 10 and FIG. 11, which illustrate activities in the use of the UCAD of the present invention in a configuration without a closing element (16) and having an insertion rod-handle (22).

In FIG. 10 the patient spreads her labia minora lips (40) with one hand and inserts a UCAD (10) into her vagina by pushing the insertion rod-handle (22) that is connected to the plate of catheter tube guiding portion (14), towards her body. The UCAD (10) in the figure is without a closing element (16) (shown in FIG. 7) and with a gap at the edge of plate of catheter tube guiding portion (14). When the UCAD (10) is in place in the body of the patient, tube (42) is inserted through hole (30) into the orifice of the urethra, as shown in FIG. 7 for an UCAD with a closing element (16).

FIG. 11 illustrates the initial activity of removing the UCAD (10) shown in FIG. 10 from the body of the patient. The illustration shows the patient pulling insertion rod-handle (22) away from her body while sliding the catheter tube guiding portion (14) along catheter tube (42). In the process, tube (42) remains in the body of the patient. In the following activity (not illustrated), at the point when the UCAD is nearly or fully removed from the body of the patient, the patient compresses and squeezes tube (42) with the fingers of her hand near the gap in hole (30) and removes the tube (42) through the gap from the UCAD, thus, leaving tube (42) in the body of the patient free from the UCAD, as illustrated in FIG. 9.

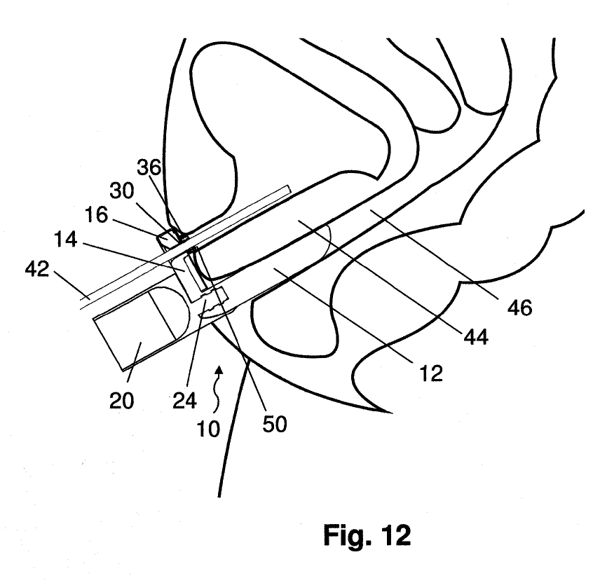

Reference is presently made to the figures FIG. 12 up to and including FIG. 13B. The figures are crosscut schematic illustrations of the positioning of the UCAD (10) of the present invention in and on the body of a treated patient.

FIG. 12 is a view from the side of the patient cross cut illustration of internal organs of a patient with the UCAD (10) deployed in the patient. The UCAD (10) is shown connected to an insertion thimble (20) and insertion portion (12) of the UCAD (10) is shown inserted into the vagina (46) and pressed against the pubic symphysis (44 of the patient. The guiding portion (14) of the UCAD (10) is pressed to the upper section of the labia minora lips (40) (shown in FIG. 13B) and hole (30) adjacent to the urethra orifice of the patient. Wing constructions (36), which are connected to guiding portion (14) and widen the urethra orifice are illustrated in FIG. 13B. Tube (42) is shown inserted through hole (30) and through the orifice of the urethra (50) of the treated patient.

FIG. 13A is a cross cut schematic illustration as viewed from the front of a patient with insertion portion (12) of UCAD (10) inserted into her vagina (as shown in FIG. 12). The illustration shows the wing constructions (36), which are connected to plate of guiding portion (14), at initiation of contact with the skin surrounding of the urethra orifice of the treated patient. FIG. 13B illustrates the wing constructions (36), shown in FIG. 13A, pressed to the skin surrounding the orifice of the urethra (50) and causing the widening of the urethra orifice (50). The widening of orifice (50) is done by pressing wings (36) to the skin surrounding the orifice and stretching it towards the labia minora lips (40). The labia minora lips (40) and labia majora lips (52) play no role in the widening of the urethra orifice (50). With the orifice widened and hole (30) of guiding portion (14) adjacent to and aligned with the orifice, catheter tube (42) is self-inserted by the patient in a manner that is easy to implement and reduces the discomfort of the insertion.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the present invention.

What is claimed:

1) A device for assisting in the insertion of a catheter tube into the external orifice of the urethral tract of women, comprising:
a vaginal insertion portion,
a catheter tube guiding portion,
at least two wing structures configured to widen the urethral orifice,
a vaginal insertion element,
wherein, said vaginal insertion portion and said catheter tube guiding portion each comprise a plate structure having an elongated configuration,
wherein, said vaginal insertion portion and said catheter tube guiding portion connect at one of their edges in a spatial configuration that forms an approximate L shaped structure,
wherein, said vaginal insertion portion has a configuration to be placed adjacent to the pubic symphysis of women.
wherein, said vaginal insertion element is connected to said vaginal insertion portion,
wherein, said catheter tube guiding portion has a hole at its unconnected edge through which said catheter tube is inserted into said orifice of urethral tract,
wherein, said wing structures connect to said catheter tube guiding portion and protrude from the rim of said hole in said catheter tube guiding portion.

2) The device as set forth in claim 1 wherein, said hole in said catheter tube guiding portion has a gap in the circumference of the hole that leads to the external surrounding of said catheter tube guiding portion.

3) The device as set forth in claim 1 wherein the device includes a hole closing bar element that reversibly connects to the edge of said catheter tube guiding portion not connected to said vaginal insertion portion,

4) The device as set forth in claim 1 and claim 3 wherein, said catheter tube guiding portion is reversibly connected to said closing bar element wherein the connection defines the said hole at the edge of said catheter tube guiding portion.

5) The device as set forth in claim 1 wherein said vaginal insertion portion and said catheter tube guiding portion of said device are produced in a connected configuration.

6) The device as set forth in claim 1 wherein, said vaginal insertion portion and said catheter tube guiding portion, each having a separate entity, connect together to conform a single entity.

7) The device as set forth in claim 1 wherein said vaginal insertion portion and said catheter tube guiding portion connect in a fixed angle of approximately ninety degrees.

8) The device as set forth in claim 1 wherein, said vaginal insertion portion and said catheter tube guiding portion connect in an adjustable fixated angle.

9) The device as set forth in claim 1 wherein said wing structures are connected to said catheter tube guiding portion in a fixed connection.

10) The device as set forth in claim 1 wherein said wing structures are connected to catheter tube guiding portion by hinges that facilitate side-movement of said wing structure.

11) The device as set forth in claim 1 wherein, said vaginal insertion element is reversibly connected to said vaginal insertion portion.

12) The device as set forth in claim 12 wherein, said vaginal insertion element is an insertion thimble.

13) The device as set forth in claim 1 wherein, said vaginal insertion element is an insertion rod-handle.
